(19) United States
(12) Patent Application Publication
(10) Pub. No.: US 2010/0063590 A1
(43) Pub. Date: Mar. 11, 2010

(54) LAMINOPLASTY IMPLANT

(75) Inventor: Andrew F. Cannestra, Jacksonville, FL (US)

Correspondence Address: WARNER NORCROSS & JUDD LLP, 900 FIFTH THIRD CENTER, 111 LYON STREET, N.W., GRAND RAPIDS, MI, 49503-2487, US

(73) Assignee: BLUE FURY CONSULTING, L.L.C., Jacksonville, FL (US)

(21) Appl. No.: 12/529,726

(22) PCT Filed: Mar. 14, 2008
(86) PCT No.: PCT/US2008/057017

§ 371 Date: Sep. 3, 2009

Related U.S. Application Data
(60) Provisional application No. 60/894,933, filed on Mar. 15, 2007.

(51) Int. Cl. A61F 2/44; A61B 17/56

(52) U.S. Cl. 623/17.11; 606/86 R

(57) ABSTRACT

A hollow non-load-bearing spinal implant having a hollow body and an attachment device for attaching the implant. A method of inserting a non-load-bearing spinal implant by inserting the laminoplasty implant into a prepared space in a spine, positioning the implant within the prepared space and affixing the implant to the spine.

The present invention relates to laminoplasty and more particularly to laminoplasty implants.

A condition associated with aging is spondylolsis, in which intervertebral disc lose water and become less dense. These degenerative changes near the disk can cause an overgrowth of the bone, producing bony spurs called, “osteophytes” that can compress the spinal cord. The constriction of the spinal cord in the cervical spine often produces pain, weakness, or loss of feeling in extremities. Other causes for narrowing of the spinal canal include disc shrinkage, which causes the disc space to narrow and the annulus to bulge and mushroom out, resulting in pressure on the spinal cord. Degenerative arthritis of facet joints can cause joints to enlarge, or the vertebra to slip with respect to each other, also compressing the spinal cord. Instability between vertebra, such as caused by stretched and thickened ligaments can also produce pressure on the spinal cord and nerve roots.

Corresponding surgical techniques can vary and will depend on many factors, including the source of the spinal cord compression, the number of vertebral segments involved in the disease process, and the cervical alignment. Two surgical methods currently exist to create additional room in the spinal canal. The first is called a laminectomy, and involves removal of the lamina (roof) of one or more vertebrae. A limitation of the laminectomy procedure is that it involves removal of the supporting structures at the back of the vertebrae which align the spinal column. The result may be that a patient suffers some postural deformity. To prevent such postural problems, a graft may be installed between the ends of the removed bone to span the void and reinstate the necessary support. The second procedure is called a laminoplasty, in which the targeted vertebra is cut, spread apart and a graft is inserted to permanently enlarge the space. Unlike the laminectomy, typically no bone material is excised during the laminoplasty procedure. Two different laminoplasty procedures are currently used. The first is called the unilateral or “open door” laminoplasty in which one side (lamina) of the vertebra is cut all the way through, while the other side is cut only half way to create a hinge. The vertebral element is then rotated about the hinge, and the graft is inserted into the opening, increasing the opening of the spinal canal. The second procedure is called the bilateral or “French door” laminoplasty in which the midline of the vertebra (spinous process) is cut all the way through, and the lamina are cut half way through, creating two hinges. The vertebral element is then opened at the bisected spinous process, and a graft inserted into the opening, again increasing the opening of the spinal canal.

Many of the currently available devices to be used in lamoinoplasty are effective, but can be difficult to implant. Another common problem with currently available implants is the lack of area in which to pack graft material. This translates to limited fusion of the bone, which controverts the intended purpose of the implant. Laminoplasty has been shown to decompress the cervical spine without the need for fusion. As such, the procedure is a motion preservation operation that does not require the use of screws across adjacent levels.

Current laminoplasty implants have significant limitations. These devices can be difficult to implant, resulting in increased medical costs. Moreover, the devices can potentially cause damage to surrounding tissue and/or to the spinal cord because of their geometry and size. The devices can limit the post-operative natural dynamics of the cervical spine, and thus can cause discomfort to the patient.

### SUMMARY OF THE INVENTION

The present invention provides a laminoplasty device, implant, or plate is hollow to provide a lattice for fusion of the lamina. The device includes fixation screw holes enabling the system to be inserted and fixed in the laminar defect similar to current laminoplasty plates. The tubular portion of the device can be packed with fusion material to promote the re-fusion of the lamina across the laminar defect. The device has an attachment point for an inserter, which will facilitate ease of placement, and may allow application during a minimally invasive procedure.

The present invention has distinct advantages over prior art laminoplasty plates. First, the hollow implant provides a structure that, when inserted into the location created during the procedure, facilitates and promotes bone fusion across a newly created laminar defect. Also, the implant promotes fusion of the lamina by enabling bone graft to be placed within the implant, which helps promote fusion, and with the use of coatings on the exterior surface that promotes bone fusion. Second, the device can be controlled easily by a single inserter with an attachment point on the device, which enables minimally invasive insertion.

These and other objects, advantages, and features of the invention will be more fully understood and appreciated by reference to the description of the current embodiment and the drawings.

### BRIEF DESCRIPTION OF THE DRAWINGS

FIG. 1 is a side elevational view of the laminoplasty implant of the present invention (the opposite side view being the minor image thereof);

FIG. 2 is a top plan view of the implant (the bottom plan view being the mirror image thereof);

FIG. 3 is a side elevational view (along laminar surface) of the laminoplasty implant of the present invention (the opposite side view being the minor image thereof);

FIG. 4 is the device holder or inserter;

FIG. 5 is a sectional view of a retractor used for insertion;

FIG. 6 is a sectional view of a cervical lamina with laminoplasty cuts;

FIG. 7 is a sectional view similar to FIG. 6 showing the insertion of the retractor in into the laminoplasty defect;

FIG. 8 is a sectional view similar to FIG. 7 showing the retractor expanded;

FIG. 9 is an elevation view of the lamina similar to FIG. 6.

FIG. 10 is an elevation view of the lamina similar to FIG. 9 showing the retractor inserted and expanded, and also showing the device to be inserted; and

FIG. 11 is an elevation view of the lamina similar to FIG. 10 showing the device inserted into the laminar defect in its final position.

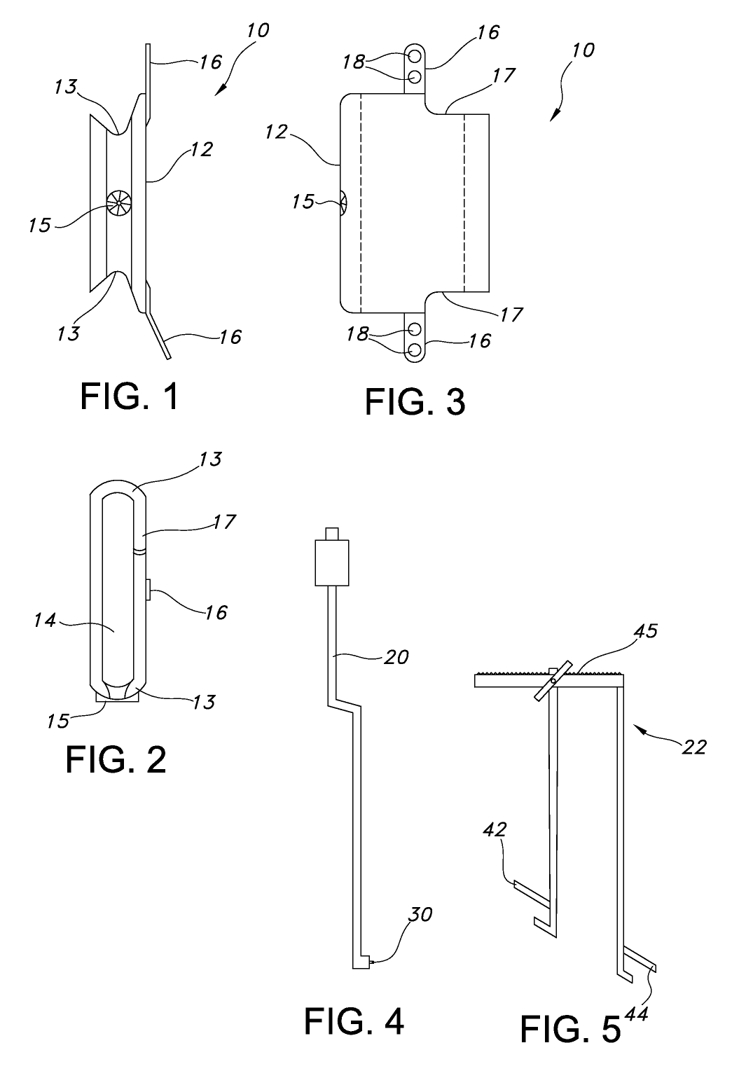

### DESCRIPTION OF THE CURRENT EMBODIMENT

The Figures illustrate one embodiment of an implant 10 according to the present invention. The implant 10 can have a variety of shapes and sizes, but preferably has a size and a geometry that enables it to be positioned in a an open door fashioned space, and to remain securely positioned while healing and fusion take place. The design can also be altered to be utilized in a bisected spinous process. Moreover, the implant 10 preferably has a substantially low profile, to prevent potential abrasion or damage to surrounding tissue. A “low profile” implant does not substantially extend outside of the cervical area. In other words, the implant does not extend outside substantially past the cervical area.

The implant can be made of any biocompatible material known to those of skill in the art to be useful in spinal implants. Examples of such materials include, but are not limited to, polyketones such as polyetheretherketone (PEEK), poly ether ketone ketone ether ketone (PEKKEK), polyethylene, or ultra high molecular weight polyethylene (UHMWPE), polyketones reinforced with biocompatible fiber fillers such as carbon, biocompatible metals, biocompatible shape memory alloys (e.g., NITINOL), a solid form of bone filler material, implantable grade alloys (including, but not limited to titanium, cobalt chrome, nitinol, or stainless steel), other medical grade composites, ceramics (including, but not limited to zirconia, alumina, or calcium-phosphate based ceramics), tissue engineered materials, chitin, urethanes, silicone, silicone materials, and resorbable polymers (for example, polylactic acid (PLA), polyglycolic acid (PGA), and poly(lactide-co-glycolide) (PLGA)), and other biocompatible materials.

The implant can be dense or porous. Porous implant can be filled with resorbable polymers to increase mechanical strength. Further, the implant can be combined with osteoinductive agents (including growth factors, cell therapy, gene therapy, and patient derived factors) and other drug therapies. The osteoinductive agents can be added to initiate and accelerate bone formation while the drug therapies can range from antibiotics to reduce the risk of infection to chemotherapy to treat cancer. Optionally, the implant can be used with a flowable bone filler material. As used herein, bone filler is defined as any substance used to stabilize the bone and includes, but is not limited to bone cement (polymethyl methacrylate (PMMA), or (PMA)), other composite material, human bone graft (allograft or autograft), synthetic and xenograft derived bone substitutes (calcium phosphate, hydroxylapatite, and/or other ceramic based bone substitutes), collagen, or combinations of these materials made of titanium, or other biocompatible materials such as stainless steel, graphite, carbon fiber materials, PEEK, nitinol, or various plastics and composites of the foregoing.

Various materials may be used for the grafts installed during laminoplasty procedures. Examples of such materials include, but are not limited to, titanium, ceramic and nylon inserts. Further, the materials can include allografts taken from long bones such as the femur, humerus, tibia and fibula. Allografts are removed from a donor and processed using known techniques to preserve the allograft until implantation. Allografts have mechanical properties which are similar to the mechanical properties of vertebrae even after processing. The benefit of such property matching is that it prevents stress shielding that occurs with metallic implants. Allografts, unlike magnetic metals, are also compatible with magnetic resonance imaging (MRI) procedures, allowing more accurate ascertainment of fusion. Furthermore, allografts are naturally osteogenic providing excellent long term fusion with the patient's own bone.

As shown in the figures, the laminoplasty implant 10 includes a body 12. The implant body 12 as shown is fabricated of a single piece of material, but could be fabricated of multiple pieces. The body 12 is formed of a tubular core 14. The tubular core 14 is hollow, which reduces the mass of the implant body 12. Additionally, the tubular core 14 enables another location for graft material to be inserted. The graft material can then diffuse out of the tubular core 14 and aid in fusion of the bone. The body 12 can further include a lattice or other devices that promote and/or facilitate fusion. The lattice is also useful when graft material is inserted into the tubular core 14, because the lattice provides a place from which the graft material can exit the tubular core 14. Examples of other devices that promote or facilitate fusion include, but are not limited to, coatings, such as those disclosed above, and other devices known to those of skill in the art to be useful in promoting and facilitating fusion of bone. The exterior surface of the body 12 can also include uneven surfaces, or gripping devices, to enable the body to better grip the surrounding bone, which promotes fusion.

The body can also include contoured portions 13. The contoured portions 13 are formed to create a snug fit between the implant 10 and the bone to which the implant 10 is affixed. More precisely, the contoured portions 13 are generally shaped to fit laminar edges, which are the edges of bone onto which the implant 10 is placed and affixed. The contoured portions 13 can also correspond to a thickening in the body 12. The thickening can be utilized to prevent wear between the bone and the implant 10.

The body 12 also includes an attachment point 15 adapted to interfit with a point 30 on an insertion device 20. The attachment point 15 can include anything that enables the implant 10 to be attached to the insertion device 20 in such a way as to maintain the implant 10 in position prior to insertion. The attachment point 15 can be a threaded attachment point. The attachment point 15 can also be easily removed from the insertion device 20 after insertion.

In another embodiment, the insertion device 20 can attached or be seated within on onto a retractor that is used to prepare the space for insertion of the device 10 of the present invention. This embodiment enables a single hand to be used by a surgeon to control both the retractor and the insertion device 20. Alternatively, the insertion device 20 can be attached to an external arm (not shown) that can stabilize the insertion device 20 when during attachment of the device 10.

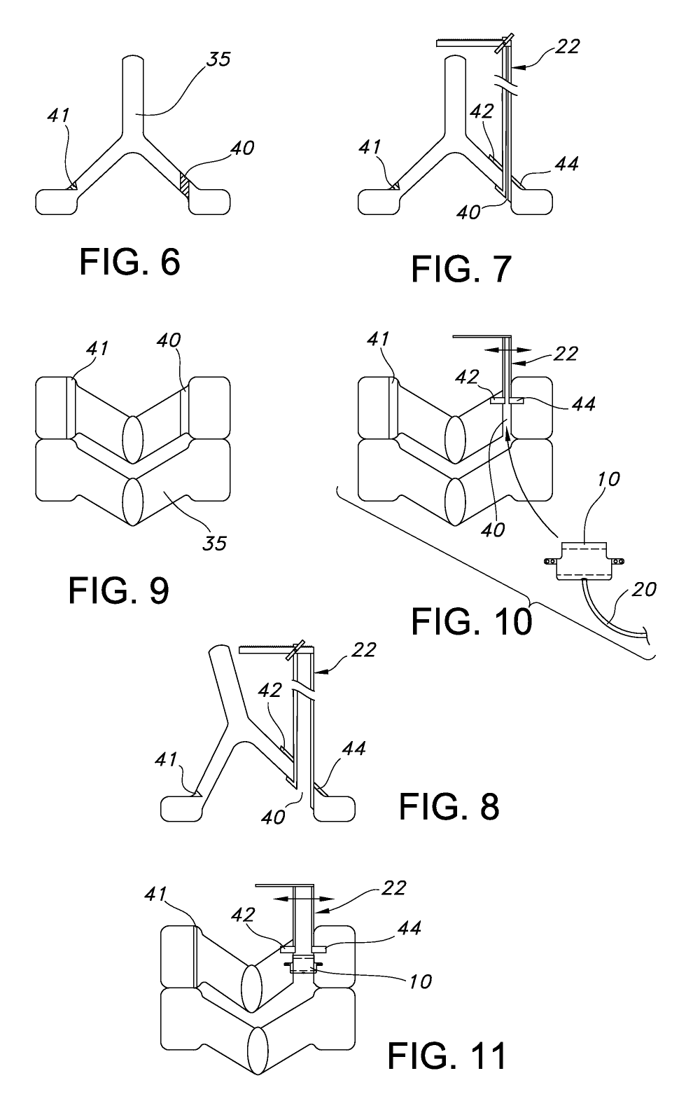

The implant 10 also includes a variety of recesses 17 that are shaped to enable the implant 10 to be positioned within the retractor 22 (see FIG. 5), enabling the implant to be inserted into a space with the retractor 22 in position (FIGS. 6-11).

The implant also includes at least one attachment device 16. The attachment device 16 can be formed as one with the body 12 or can be separate from the body 12 and affixed to the body 12. The attachment device 16 can be flexible or malleable, enabling the attachment device 16 to be properly oriented and positioned during the procedure. The attachment device 16 can be formed of the same material as the body 12 or of a material that is compatible with the material of the body 12. In other words, the material that is used to form the attachment device 16 cannot be a material that can denigrate or render useless the body 12 after attachment of the attachment device 16 to the body 12. The attachment device 16 can be any device that is able to be manipulated into place and subsequently used to affix the body 12 in place within the spine. In the embodiment shown in the Figures, the attachment device 12 is a pair of opposite anchor tabs or fixation points. The tabs 16 extend outward from opposite sides of the body 12. In the figures the tabs 16 are positioned at the mid portion of the body 12, however, the position can be altered depending on the size of the implant 10 and site to which the implant 10 is being inserted.

The attachment device 16 includes at least one aperture 18 through which at least one anchoring device (not shown) is placed. The apertures 18 enable the placement of an anchoring device (not shown) into the bone thereby fixing the implant 10 in place. As shown in the figures, the anchoring device can be bone screws or other similar devices known to those of skill in the art. The aperture 18 is sized to enable the screw or other anchoring device to pass therethrough, but not substantially larger. By keeping the aperture close in size to the anchoring device, there is a limit in the amount of movement of the anchoring device. Examples of anchoring devices include, but are not limited to, bone screws, and other removable, biocompatible devices.

### Installation/Use

Hardware and the procedure for inserting the laminoplasty implant 10 within a laminoplasty defect space are illustrated in FIGS. 6-11. The laminoplasty implant 10 is inserted using an inserter 20 (FIG. 4) with an attachment point 30 to mate with the attachment point 15.

A laminoplasty is performed by cutting or drilling a trough 40 along the lamina-facet junction of the spine 35 and by creating a living hinge 41 along the lamina-facet junction on the opposite side of the spine 35 (see FIGS. 6 and 9). The retractor 22 (FIG. 5) is placed within the trough (laminoplasty) defect (FIG. 7) and expanded (see FIGS. 8 and 10). The retractor includes an upper stop 42 and a lower stop 44 to limit the travel of the retractor into the spinal canal. The stops 42 and 44 are positioned between the laminar edges at the defect (FIG. 7) and then the retractor 22 is expanded using a ratcheting mechanism 45 to create a larger laminoplasty defect (FIGS. 8 and 10).

The implant 10 is then placed within the defect using the inserter 20 (FIGS. 10-11) and secured into place using screws (not shown). The retractor 22 is then removed (not shown) and the implant is maintained in position.

The present invention provides a medical implant device for use in spinal surgery, and more preferably for use in laminoplasty surgery. The implant can be a laminoplasty cage having any cage implant shape known to those of skill in the art, having a generally hollow, elongate body with open ends. The implant can be adapted for use in a variety of applications, but is preferably used to maintain the position of vertebra after midline or open door laminoplasty surgery. The implant is particularly advantageous in that it is easy to implant, it enables permanent bony incorporation when used both with and without bone growth promoting materials. It enables muscle re-attachment. It enables the natural dynamics of the cervical spine. It has a substantially low-profile, such that is does not extend substantially outside of the spinal cord and into the surrounding tissue, to avoid or prevent damage to surrounding tissue.

The above description is that of the current embodiment of the invention. Various alterations and changes can be made without departing from the spirit and broader aspects of the invention as defined in the appended claims, which are to be interpreted in accordance with the principles of patent law including the doctrine of equivalents. Any reference to a claim element in the singular, for example, using the articles “a,” “an,” “the” or “said,” is not to be construed as limiting the element to the singular.

The embodiments of the invention in which an exclusive property or privilege is claimed are defined as follows:

1. A hollow non-load-bearing spinal implant comprising:
a hollow body;
attachment means integral within said body for attaching the implant.

2. The implant according to claim 1, wherein said body is tubular.

3. The implant according to claim 2, wherein said body is formed of a material selected from the group consisting essentially of polyketones, poly ether ketone ketone ether ketone, polyethylene, and ultra high molecular weight poly ethylene.

4. The implant according to claim 1, wherein said body includes a coating on an exterior surface of said body.

5. The implant according to claim 1, wherein said implant is a laminoplasty implant.

6. The implant according to claim 1, wherein said body includes a lattice for promoting bone fusion.

7. The implant according to claim 1, wherein said body includes contouring means for fitting the implant in close proximity to bone.

8. The implant according to claim 1, wherein said body is formed of at least one piece of material.

9. The implant according to claim 1, wherein said body includes retention means for retaining said body within an insertion device.

10. The implant according to claim 1, wherein said attachment means includes material extending from said body enabling attachment of said body to bone.

11. The implant according to claim 10, wherein said material is a tab.

12. The implant according to claim 11, wherein said tabs are malleable.

13. The implant according to claim 10, wherein said attachment means has an uneven exterior for gripping or otherwise maintaining said body in position.

14. The implant according to claim 10, wherein said attachment means includes screw receiving means for receiving and holding a screw in place therein.

15. A method of inserting a hollow non-load-bearing spinal implant by:
inserting an insertion device into a prepared space in a spine;
positioning the hollow non-load-bearing spinal implant within the prepared space; and
affixing the implant to the spine.

16. The method according to claim 15, further including preparing the space in need of the spinal implant.

17. The method according to claim 16, wherein said preparing step includes:
drilling a trough along the lamina-facet;
inserting a retracting into the trough; and
expanding the trough space with the retractor to prepare the space for insertion of the implant.
